(19) United States
(12) Patent Application Publication
(10) Pub. No.: US 2008/0317871 A1
(43) Pub. Date: Dec. 25, 2008

(54) Use of Hydrochloric Acid For the Manufacture of a Medicament For the Treatment of Hypertension

(76) Inventor: Zhiren Liu, Zhejiang (CN)

Correspondence Address: HESLIN ROTHENBERG FARLEY & MESITI PC, 5 COLUMBIA CIRCLE, ALBANY, NY, 12203, US

(21) Appl. No.: 11/908,053

(22) PCT Filed: Mar. 6, 2006
(86) PCT No.: PCT/CN06/00329

§ 371 Date: Sep. 7, 2007

(30) Foreign Application Priority Data
Mar. 10, 2005 (CN) 200510049329.8

(51) Int. Cl. A61K 33/20; A61P 7/00

(52) U.S. Cl. 424/666

(57) ABSTRACT

A method of treating hypertension by administering to a patient an aqueous solution of HCl having a pH from 3 to 6.9 for a period of from 0.5 to 6 years and a method of treating a calculus disease by administering an aqueous solution of HCl having a pH from 3 to 6.9 are disclosed.

### FIELD OF THE INVENTION

The present invention relates to the use hydrochloric acid (HCl) for the treatment of hypertension as well as calculus.

### BACKGROUND OF THE INVENTION

Many factors are responsible for human hypertension, including predisposition, endocrine disorder and bad diet. It is difficult to find a method to cure hypertension. Thus the medical community thinks hypertension is incurable and requires patients to take anti-hypertension drugs for their lifetime. Nowadays all drugs and instruments used to treat hypertension are intended to dilate arterial vessels, causing faster aging of the vessels due to recurrent dilation. Even worse, dropping out of debris from the walls of the vessels due to dilation of the vessels may cause myocardial infarction.

If hypertension is heritable, a newborn should be a hypertension patient, but this kind of newborn is hard to find. If hypertension is caused by bad diet, the question arises as to why no patient has been cured by changing his/her diet so far. This evidence runs against the idea that patients suffering from hypertension should take anti-hypertension drugs for their whole lifetime. Meanwhile, if hypertension is caused by aged and hardened arterial vessels, merely using drugs or instruments will not dilate the vessels. Thus the idea that hypertension is caused by aged and hardened arterial vessels is untenable.

### SUMMARY OF THE INVENTION

The present invention is to use HCl to prepare a medicament for the treatment of hypertension.

Inventors of the present invention after many years' endeavor find the mechanism of hypertension is as below: 1) Calcium bicarbonate in water and food can decompose forming binders such as calcium carbonate and calcium oxalate. The binders can attach human metabolites (such as crystallized cholesterol) and other solid materials that enter into the human body onto the inner walls of arterial and capillary vessels. With age, the attached materials on the inner walls become thicker and thicker and the aperture of the vessels become narrower and narrower, blocking blood flow and increasing blood pressure.

In addition, deposition of calcium carbonate and calcium oxalate in the kidneys, liver and the gallbladder results in renal, hepatic and biliary calculus.

2) Materials attached to the inner walls of the vessels reduce the flexibility of the vessels, preventing self-modulation of blood pressure;

3) The long attachment of the materials to the inner walls of the vessels insidiously destroys blood vessels, reducing flexibility of the vessels.

Thus if the materials that attached to the inner walls of the vessels can be dissolved, blood pressure can be restored to normal. To dissolve the attached materials, the calcium carbonate and calcium oxalate should be dissolved, releasing and discharging other small particles. The inventors of the present invention find that HCl is the best material to dissolve calcium carbonate and calcium oxalate. Meanwhile, HCl can be absorbed to inner walls by the protective membranes of the blood vessels, thus repairing the damaged vessels and restoring their original elasticity.

In addition, the inventors of the present invention find that HCl is able to help dissolve renal, hepatic and biliary calculus alleviating or removing symptoms of calculus.

In accordance with one aspect of the invention there is provided a method of using HCl to manufacture anti-hypertension medicines.

In another preferred embodiment, an aqueous solution with pH in the range of 3 to 6.9 (4-6.5 is preferable) is used.

In another preferred embodiment, the pH of the aqueous solution is in the range of 6.5 to 6.9.

In another preferred embodiment, the drug is in the form of an oral formulation.

In another preferred embodiment, the drug is in the form of an injectable formulation.

In another preferred embodiment, the drug is an aqueous solution of HCl having a pH of 3-6.9 (4-6.5 is preferable), and the aqueous solution meets drinkable water requirements.

In another preferred embodiment, the drug is an aqueous solution of HCl having a pH of 3-6.9, and the aqueous solution contains extra anti-hypertension medications that can keep stable within the above pH ranges.

According to another aspect of the invention there is provided a method to treat hypertension, i.e., administer to subjects 100-3000 ml of HCl aqueous solution of pH 3-6.9 each day for 0.5-6 years.

Preferably, the pH range of the aqueous solution is 4-6.5 or 6.5-6.9.

According to another aspect of the invention there is provided a kind of anti-hypertension formulation, which is an HCl aqueous solution of pH 3-6.9, and the aqueous solution contains extra anti-hypertension medications that can keep stable within the above pH ranges.

In another preferred embodiment, the extra anti-hypertension medications are selected from the group consisting of, but not limited to, Amlodipine, Benazepril, Captopril, Clonidine, Enalaprilat, Felodipin, Guanazodine, Indanidine, Indapamide, Minoxidil, Nitrendipine, Perindopril, Proroxan, Reserpiline, Trimoxamine, Tripamide, Utibapril, Zabiciprilat or combinations thereof.

Preferably, the formulation is an injectable formulation.

According to yet another aspect of the invention, this invention provides HCl to prepare medicines for treating calculus.

In another preferred embodiment, the calculus diseases are selected from the group consisting renal calculus, hepatic calculus or biliary calculus.

The drug in the present invention is usually an aqueous solution that contains HCl when this invention uses HCl to prepare medications to treat hypertension or calculus, and the pH of the aqueous solution is in the range of 3 to 6.9 (4-6.5 is preferable). China's national standard requires drinkable water to have a pH of 6.5-8.5, indicating that the aqueous solution that contains HCl can be directly drunk as potable HCl water if the above aqueous solution is adjusted to have a pH between 6.5 and 6.9.

The drug can be prepared for oral formulation, or injectable formulation, or other formulation forms. Oral intake of HCl aqueous solution should not be less than 1500 ml each day if it is used to treat hypertension or calculus, and in three to six divided doses a day and 3-6 months make up a therapeutic duration. If the solution is injected for treating hypertension, the recommended daily dosage is 500 ml, two divided intravenous doses, 2-3 months make up a therapeutic duration. If the above method and dosage are adopted to treat hypertension, one year later the dosage can be reduced by a quarter. The dosage can be reduced by a half two years later, and three years later, the patient merely needs a maintenance dosage or can be relieved from taking anti-hypertension medications but maintain a normal blood pressure.

The HCl aqueous solution for oral use can be artificially prepared according to the following method: pour HCl into drinkable water and stir, precisely test pH before filling containers for disinfection. Keep pH between 4 and 6.9. For potable HCl aqueous solutions, the pH should be controlled between 6.5 and 6.9.

In industrial situations, the HCl aqueous solution can be mass manufactured continuously and stably using a pH controller apparatus.

The HCl injection formulation can be made according to common pharmaceutical methodology only if the pH of the injectable is controlled between 3 and 6.9 (4-6.5 is preferable).

In case tap water is used to prepare HCl water, the beginning part of the production line should be equipped with a water purification apparatus ensuring the water inlet to meet national drinking-water standards.

To treat hypertension, the HCl of the present invention can be used with other solutions such as physiological salt solutions, glucose solutions and beverages, sharing the common method with the above only if pH is controlled as required.

The potable HCl water described by the present invention should not be heated for drinking because heating will evaporate HCl and the solution will lose therapeutic effect. Beverages containing binding materials and tea should be avoided because the base materials can neutralize HCl.

Evidence of the therapeutic effect of HCl for hypertension of the present invention was obtained by clinical observations of 126 hypertension patients who drank the HCl aqueous solution. The subjects included 87 male patients and 39 female. The average age of the patients was 68.2 and ranged from 51-81 years old.

The results indicated that 89 patients were cured by drinking HCl water in combination with anti-hypertension medications and exclusively drinking potable HCl water; 30 patients had their blood pressure restored to normal though halving their anti-hypertension drug dosage in combination with drinking HCl water; and 7 patients obtained normal blood pressure after having drunk potable HCl water for two years. Previously, before they took HCl water, they were unable to have normal blood pressure although they took anti-hypertension medications for the same amount of time that they took HCl water.

The present invention also observed patients suffering from renal, hepatic and/or biliary calculus, and the results indicate that drinking HCl water helps alleviate or remove calculus.

Medications that are prepared from HCl and for treatment of hypertension and calculus described by the present invention are safe and reliable, and have credible and obvious effects. They can also significantly relieve hypertension patients with hardened arterial vessels, and can modify harmful calcium salts to provide calcium needed by the human body.

### DETAILED DESCRIPTION OF THE EMBODIMENTS

Hereinafter, the present invention will be described more specifically with reference to the following Examples. The following Examples are for illustrative purpose and are not intended to limit the scope of the invention. For Examples that have not been attached with concrete condition for the experimental methods, the acknowledged condition or condition denoted by manufacturers can be referred to. Portion or percentage refers to weight portion or weight percentage except for those that have been specially remarked.

### Example 1

Pour highly purified dilute HCl into drinkable water and stir until homogenous, precisely control pH to 6.5-7. This kind of solution can be directly taken orally.

### Example 2

Pour highly purified HCl into drinkable water to form 2% diluted HCl solution. The solution is then poured in a vessel equipped with a flow volume controller. Mix the HCl solution and drinkable water via pipes at a certain volume ratio, and at the end part of the pipe a pH detector is installed. Keeping the pH of the HCl aqueous solution in the range of 6.5 to 6.9, thus obtains potable HCl water.

The above potable HCl water is administered to 16 hypertension patients at a daily dosage of 2000 ml. The patients took 500 ml one hour before breakfast and lunch respectively, and 1000 ml before supper.

Table 1 demonstrates alleviation of hypertension after having taken edible HCl water.

TABLE 1
Alleviation of hypertension after having taken edible HCl water.

| | Original | | |
|---|---|---|---|
| Patient | blood | | |
| number | pressure | Age | Therapeutic effect |
| 1 | 130/110 | 51 | The patient halved dosage one year later, and |
| | | | stopped using anti-hypertension drugs in July 2002 |
| | | | for 2 years to date, the patient now does not use |
| | | | any anti-hypertension drugs, and has a blood |
| | | | pressure of 120/80. |
| 2 | 180/110 | 75 | After the patient had been drinking potable HCl |
| | | | water for one and a half year, the patient halved his |
| | | | dosage and his blood pressure was stable. Three |
| | | | years later the patient stopped using |
| | | | anti-hypertension drugs, and the blood pressure |
| | | | was 110/70, the patient has abandoned |
| | | | anti-hypertension drugs for two years and the |
| | | | blood pressure keeps stable at 110/70. |
| 3 | 180/95 | 74 | The patient stopped using anti-hypertension drugs |
| | | | after he had been drinking HCl water for three |
| | | | years, and the blood pressure was 130/80. The |
| | | | patient has stopped using anti-hypertension drugs |
| | | | for two years, and the blood pressure keeps stable at 130/80. |
| 4 | 180/105 | 71 | The patient stopped using anti-hypertension drugs |
| | | | after he had been drinking HCl water for one and a |
| | | | half years, his blood pressure is 130/80. He has |
| | | | abandoned anti-hypertension drugs for two years |
| | | | and his blood pressure keeps stable at 130/80. |
| 5 | 150/90 | 75 | The patient stopped using anti-hypertension drugs |
| | | | after he had been drinking HCl water for three |
| | | | years. He has abandoned anti-hypertension drugs |
| | | | for two years and his blood pressure keeps stable at 130/85. |
| 6 | 200/100 | 82 | The patient halved his anti-hypertension drugs |
| | | | dosage after he had been drinking HCl water for |
| | | | one year, his blood pressure is 140/90. |
| 7 | 160/95 | 75 | The patient stopped using anti-hypertension drugs |
| | | | after he had been drinking HCl water for three |
| | | | years. He has abandoned anti-hypertension drugs |
| | | | for one and a half years and his blood pressure is 135/85. |
| 8 | 190/85 | 79 | The patient halved his anti-hypertension drugs |
| | | | after he had been drinking HCl water for three |
| | | | years, and his blood pressure is 130/80. |
| 9 | 150/90 | 73 | The patient stopped using anti-hypertension drugs |
| | | | after he had been drinking HCl water for two |
| | | | years. He had abandoned anti-hypertension drugs |
| | | | for one and a half years and his blood pressure is 133/73. |
| 10 | 160/100 | 74 | The patient stopped using anti-hypertension drugs |
| | | | after he had been drinking HCl water for three |
| | | | years. He has abandoned anti-hypertension drugs |
| | | | for two years and his blood pressure is 120/80. |
| 11 | 180/95 | 66 | The patient stopped using anti-hypertension drugs |
| | | | after he had been drinking HCl water for four |
| | | | years. He had abandoned anti-hypertension drugs |
| | | | for one year and his blood pressure is 120/80. |
| 12 | 180/110 | 72 | The patient halved his anti-hypertension drugs |
| | | | after he had been drinking HCl water for two years |
| | | | and his blood pressure is 128/65. |
| 13 | 170/110 | 62 | The patient stopped using anti-hypertension drugs |
| | | | after he had been drinking HCl water for three |
| | | | years. He has abandoned anti-hypertension drugs |
| | | | for one year and his blood pressure is 140/90. |
| 14 | 160/98 | 74 | The patient stopped using anti-hypertension drugs |
| | | | after he had been drinking HCl water for three |
| | | | years. He has abandoned anti-hypertension drugs |
| | | | for three years and his blood pressure is 135/80. |
| 15 | 160/100 | 63 | The patient stopped using anti-hypertension drugs |
| | | | after he had been drinking HCl water for three |
| | | | years. He has abandoned anti-hypertension drugs |
| | | | for two years and his blood pressure is 125/80. |
| 16 | 145/95 | 70 | The patient stopped using anti-hypertension drugs |
| | | | after he had been drinking HCl water for two |
| | | | years. He has abandoned anti-hypertension drugs |
| | | | for two years and his blood pressure is 120/75. |

Table 1 illustrates that HCl aqueous solution is very effective in treating hypertension with a total effectiveness rate of 100%.

### Example 3

Pour highly purified HCl into drinkable water to form 2% diluted HCl solution. The solution is then poured in a vessel equipped with a flow volume controller. Mix the HCl solution and drinkable water using pipes at a certain volume ratio, and at the end part of the pipe a pH detector is installed. Keeping the pH of the HCl aqueous solution in the range of 4.0 to 6.5, thus obtain potable HCl water.

The above potable HCl water is administered to patients suffering from calculus at a daily dosage of 2000 ml given the HCl water has a pH 6.5. The patients took 500 ml one hour before breakfast and lunch respectively, and 1000 ml before supper. In case the pH of the HCl water is lower than 6.5, the volume of water can be reduced. For instance, given the HCl water has a pH 5.0, the daily dosage can be reduced to 1000 ml, and the patients took 250 ml one hour before breakfast and lunch respectively, and 500 ml before supper.

Table 2 and 3 demonstrate alleviation of calculus after having taken potable HCl water.

TABLE 2
Alleviation of renal calculus after having taken edible HCl water.

| | Diameter of | | |
|---|---|---|---|
| | calculus in | | |
| Name | kidney (cm) | Age | Therapeutic effect |
| Zhang | 0.9 | 77 | The diameter became 0.48 after the patient had been |
| | | | drinking HCl water for one year and the calculus |
| | | | disappeared two years later |
| Ju | 0.41 | 70 | The calculus disappeared after the patient had been |
| | | | drinking HCl water for one year. |
| Mei | 0.9 in left | 29 | The diameter became 0.5 (left kidney) and 0.45 |
| | kidney, and | | (right kidney) after the patient had been drinking |
| | 0.8 in right | | HCl water for one year, and the calculus disappeared |
| | kidney | | two years later. |
| Lin | 0.52 | 46 | The calculus disappeared after the patient had been |
| | | | drinking HCl water for one year. |
| Han | 0.8 | 63 | The diameter became 0.5 after the patient had been |
| | | | drinking HCl water for one year and the calculus |
| | | | disappeared one and a half years later. |
| Zhou | 1.2 | 52 | The diameter became 0.8 after the patient had been |
| | | | drinking HCL water for one year and disappeared |
| | | | two years later. |
| Huang | 0.95 | 38 | The diameter became 0.52 after the patient had been |
| | | | drinking HCL water for one year and the calculus |
| | | | disappeared two years later |
| Gao | 0.6 | 45 | The calculus disappeared after the patient had been |
| | | | drinking HCl water for one year. |
| Zhang | 0.7 | 21 | The calculus disappeared after the patient had been |
| | | | drinking HCl water for ten months. |
| Cao | 0.65 | 62 | The diameter became 0.5 after the patient had been |
| | | | drinking HCl water for a half year and the calculus |
| | | | disappeared one and a half years later. |

TABLE 3
Alleviation of hepatic and biliary calculus after having taken edible HCl water.

| | Diameter | | |
|---|---|---|---|
| | of calculus | | |
| | in liver and | | |
| | gallbladder | | |
| Name | (cm) | Age | Therapeutic effect |
| Tan | 0.7 × 0.6 | 63 | The calculus was cured after the patient had been |
| | | | drinking HCl water for ten months (confirmed by |
| | | | B-type ultrasound inspection). |
| Ying | 0.7 | 53 | The diameter became 0.4 after the patient had been |
| | | | drinking HCl water for one year, and the calculus |
| | | | disappeared one and a half year later. |
| Xu | 0.8 | 74 | The calculus disappeared after the patient had been |
| | | | drinking HCl water for two years. |
| Zhang | 1.6 | 69 | The diameter became 1.2 after the patient had been |
| | | | drinking HCl water for one year, and became 0.6 |
| | | | after the patient had been drinking HCl water for |
| | | | two years, the calculus disappeared two and a half |
| | | | years later. |
| Lu | Two stones | 75 | The diameter of two stones turned to be 0.7 after |
| | have a | | the patient had been drinking HCl water for one |
| | diameter of | | year, and the calculus disappeared two and a half |
| | 1.5 and one | | years later. |
| | stone has a | | |
| | diameter of | | |
| | 0.8 | | |
| Chen | Φ1.3 | 52 | The diameter became 0.8 after the patient had been |
| | | | drinking HCL water for one year (confirmed by |
| | | | B-type ultrasound inspection). |
| Liu | Φ1.8 | 65 | The diameter became 1.1 after the patient had been |
| | | | drinking HCL water for one year and turned to be |
| | | | 0.6 after the patient had been drinking HCL water |
| | | | for two years. |
| Liu | Φ1.1 | 62 | The calculus disappeared after the patient had been |
| | | | drinking HCl water for one and a half years. |
| Ni | Φ1.9 × 5 | 55 | The diameter became 1.6 × 4.1 after the patient had |
| | | | been drinking HCL water for one year and turned |
| | | | to be 1.6 × 3.1 after the patient had been drinking |
| | | | HCL water for two years. |
| Zhu | Φ2.1 | 67 | The diameter became 1.1 after the patient had been |
| | | | drinking HCl water for one and a half years. |

Table 2 and 3 illustrate that HCl water is significantly effective in treating renal, hepatic and biliary calculus.

### Example 4

A compound formulation consisting of HCl aqueous solution and anti-hypertension drugs.

Add one tablet (2.5 mg) of market available Indapamide into 500 ml of HCl aqueous solution of pH 6.9, making up a compound formulation that contains HCl and Indapamide.

Similarly, other compound formulations that contain HCl and anti-hypertension drugs can be obtained.

All the references mentioned in this specification are herein incorporated into the specification, to the same extent as if each individual reference was specifically and individually indicated to be incorporated herein by reference. It should be understood that after having reviewed the above contents of the present invention, those ordinary skilled of the art can make variations and modifications to the present invention, which are still within the spirit and scope of the appended claims.

1-10. (canceled)

11. A method of treating hypertension comprising administering to a patient an aqueous solution of HCl having a pH from 3 to 6.9 for a period of from 0.5 to 6 years.

12. A method according to claim 11, wherein the patient was administered between 100 and 300 ml of the aqueous solution of HCl every day.

13. A method according to claim 11, wherein the pH is from 6.5 to 6.9.

14. A method according to claim 11, wherein the aqueous solution of HCl is in the form of an oral formulation.

15. A method according to claim 11, wherein the aqueous solution of HCl in the form of an injectable formulation.

16. A method according to claim 11, wherein the aqueous HCl solution further comprises an anti-hypertension medication, the anti-hypertension medication being capable of maintaining its stability in the aqueous solution of HCl.

17. A method according to claim 16, wherein the anti-hypertension medication is chosen from Amlodipine, Benazepril, Captopril, Clonidine, Enalaprilat, Felodipin, Guanazodine, Indanidine, Indapamide, Minoxidil, Nitrendipine, Perindopril, Proroxan, Reserpiline, Trimoxamine, Tripamide, Utibapril, Zabiciprilat, and combinations thereof.

18. A method of treating a calculus disease comprising administering an aqueous solution of HCl having a pH from 3 to 6.9.

19. A method according to claim 18, where the calculus disease is selected from renal calculus, hepatic calculus, biliary calculus, and combinations thereof.
